(19) United States
(12) Patent Application Publication
(10) Pub. No.: US 2019/0150764 A1
(43) Pub. Date: May 23, 2019

(54) System and Method for Estimating Perfusion Parameters Using Medical Imaging

(71) Applicant: The Regents of the University of California, Oakland, CA (US)

(72) Inventors: Corey Arnold, Oakland, CA (US); King Chung Ho, Oakland, CA (US); Fabien Scalzo, Oakland, CA (US)

(21) Appl. No.: 16/098,482

(22) PCT Filed: May 2, 2017
(86) PCT No.: PCT/US2017/030698

§ 371 Date: Nov. 2, 2018

Related U.S. Application Data
(60) Provisional application No. 62/330,773, filed on May 2, 2016.

(51) Int. Cl. A61B 5/026; A61B 5/0295; A61B 5/00; A61B 5/0285; G06K 9/66; G06T 7/00; G16H 30/40; G16H 15/00

(52) U.S. Cl. CPC A61B 5/0263; G06T 2207/30104; A61B 5/7267; A61B 5/0285; A61B 5/0042; G06K 9/66; G06T 7/0016; G16H 30/40; G16H 15/00; A61B 2576/026; G06K 2209/05; G06T 2207/10096; G06T 2207/20081; G06T 2207/20084; A61B 5/0295

(57) ABSTRACT

A system and method for estimating perfusion parameters using medical imaging is provided. In one aspect, the method includes receiving a perfusion imaging dataset acquired from a subject using an imaging system, and assembling for a selected voxel in the perfusion imaging dataset a perfusion patch that extends in at least two spatial dimensions around the selected voxel and time. The method also includes correlating the perfusion patch with an arterial input function (AIF) patch corresponding to the selected voxel, and estimating at least one perfusion parameter for the selected voxel by propagating the perfusion patch and AIF patch through a trained convolutional neural network (CNN) that is configured to receive a pair of inputs. The method further includes generating a report indicative of the at least one perfusion parameter estimated.

### CROSS-REFERENCE TO RELATED APPLICATIONS

This application is based on, claims priority to, and incorporates herein by reference in their entirety U.S. Ser. No. 62/330,773 filed May 2, 2016, and entitled “METHOD AND APPARATUS FOR ESTIMATING PERFUSION MAPS FROM MAGNETIC RESONANCE (MR) PERFUSION WEIGHTED IMAGES.”

### STATEMENT OF FEDERALLY SPONSORED RESEARCH

This invention was made with government support under NS076534 awarded by the National Institutes of Health. The government has certain rights in the invention.

### BACKGROUND

The present disclosure relates generally to medical imaging. More particularly, the present disclosure is directed to systems and methods for analyzing perfusion imaging.

Any nucleus that possesses a magnetic moment attempts to align itself with the direction of the magnetic field in which it is located. In doing so, however, the nucleus precesses around this direction at a characteristic angular frequency (Larmor frequency), which is dependent on the strength of the magnetic field and on the properties of the specific nuclear species (the magnetogyric constant γ of the nucleus). Nuclei which exhibit these phenomena are referred to herein as “spins.”

When a substance such as human tissue is subjected to a uniform magnetic field (polarizing field B₀), the individual magnetic moments of the spins in the tissue attempt to align with this polarizing field, but precess about it in random order at their characteristic Larmor frequency. A net magnetic moment M_{Z} is produced in the direction of the polarizing field, but the randomly oriented magnetic components in the perpendicular, or transverse, plane (x-y plane) cancel one another. If, however, the substance, or tissue, is subjected to a transient electromagnetic pulse (excitation field B₁) which is in the x-y plane and which is near the Larmor frequency, the net aligned moment, Mz, may be rotated, or “tipped”, into the x-y plane to produce a net transverse magnetic moment Mₜ, which is rotating, or spinning, in the x-y plane at the Larmor frequency. The practical value of this phenomenon resides on signals that are emitted by the excited spins after the pulsed excitation signal B₁ is terminated. Depending upon chemically and biologically determined variable parameters such as proton density, longitudinal relaxation time (“T1”) describing the recovery of M_{Z} along the polarizing field, and transverse relaxation time (“T2”) describing the decay of Mt in the x-y plane, this nuclear magnetic resonance (“NMR”) phenomena is exploited to obtain image contrast and concentrations of chemical entities or metabolites using different measurement sequences and by changing imaging parameters.

When utilizing NMR to produce images and chemical spectra, a technique is employed to obtain NMR signals from specific locations in the subject. Typically, the region to be imaged (region of interest) is scanned using a sequence of NMR measurement cycles that vary according to the particular localization method being used. To perform such a scan, NMR signals from specific locations in the subject are obtained by employing magnetic fields (Gx, Gy, and Gz) which have the same direction as the polarizing field B₀, but which have a gradient along the respective x, y and z axes. By controlling the strength of these gradients during each NMR cycle, the spatial distribution of spin excitation can be controlled and the location of the resulting NMR signals can be identified from the Larmor frequencies typical of the local field. The acquisition of the NMR signals is referred to as sampling k-space, and a scan is completed when sufficient NMR cycles are performed to fully or partially sample k-space. The resulting set of received NMR signals are digitized and processed to reconstruct the image using various reconstruction techniques.

To generate an MR anatomic image, gradient pulses are typically applied along the x, y and z-axis directions to localize the spins along the three spatial dimensions, and MR signals are acquired in the presence of one or more readout gradient pulses. An image depicting the spatial distribution of a particular nucleus in a region of interest of the object is then generated, using various post-processing techniques. Typically, the hydrogen nucleus (1H) is imaged, though other MR-detectable nuclei may also be used to generate images.

Stroke is the second most common cause of death worldwide and remains a leading cause of long-term disability. Recanalization of the occluded vessel is the objective of current therapies and can lead to recovery if it is achieved early enough. However, recanalization is also associated with higher risks of hemorrhagic transformation especially in the context of poor collateral flow and longer time to treatment. While safety time windows have been established based on population studies, a given individual patient may be unnecessarily excluded from a high-impact treatment opportunity.

MR imaging, and more specifically perfusion-weighted MR imaging, is a common modality used in the diagnosis and treatment of patients with brain pathologies, such as stroke or cancer. Specifically, perfusion-weighted images (“PWI”) are typically obtained by injecting a contrast bolus, such as a gadolinium chelate, into a patient's bloodstream. Images are then acquired as the bolus passes through the patient using dynamic susceptibility contrast (“DSC”) or dynamic contrast enhanced (“DCE”) techniques. The susceptibility effect of the paramagnetic contrast leads to signal loss that can be used to track contrast concentration in specific tissues over time. By applying various models to the resulting concentration-time curves, a number of perfusion parameters can be determined, such as blood volume (“BV”), blood flow (“BF”), mean transit time (“MTT”), time-to-peak (“TIP”), time-to-maximum (“Tmax”), maximum signal reduction (“MSR”), first moment (“FM”), and others. These can be used to determine a chronic or acute condition of the patient. For example, Tmax and MTT have been used to predict a risk of infarction.

Typically, deconvolution algorithms, such as the single value decomposition (“SVD”), are utilized to generate perfusion parameters from PWI. In these approaches, the measured concentration-time curve (“CTC”) of a region of interest (“ROI”) is expressed as the convolution between an arterial input function (“AIF”) and a residual (“R”) function, as shown in FIG. 1. Specifically, the AIF describes the contrast input in the voxel or ROI, while the R function expresses the residual amount of contrast in the voxel or ROI. Different curve features may then be used to estimate various perfusion parameters, as indicated in FIG. 1.

However, there are growing concerns that perfusion parameters obtained using such deconvolution techniques are less predictive due to errors and distortions introduced during the deconvolution process. This is because the acquired concentration curves are generally very noisy, and the deconvolution may produce residue functions that are not physiologically plausible. In addition, values for the generated parameters, and hence conclusions drawn thereupon, can vary depending upon the specific models and model assumptions utilized. Recognizing these limitations, several groups have attempted to develop alternative techniques aiming to provide more robust estimate of perfusion parameters. For example, delayed-corrected SVD (dSVD) was developed to perform deconvolution while doing delay correction for contrast delay. Another common delay-insensitive method includes the block-circulant SVD (bSVD), which employs a block-circulant decomposition matrix to remove the causality assumption built into standard SVD. Additionally, an oscillation index has been used as a threshold in an iterative process of repeating bSVD deconvolution to identify the best residue function, known as oscillation-index SVD (oSVD). Other approaches include the Gaussian Process deconvolution, which applies Gaussian priors for individual time points to produce a smoother estimate for the residue function. Smoother residue functions have also been obtained using Tikhonov regularization, where an oscillation penalty is applied in a least squares solution or using Gamma-variate functions. Yet other approaches have included Bayesian estimation of perfusion parameters that could handle higher levels of noise at the cost of longer computation times.

In light of the above, there is a need for improved image analysis techniques that can provide accurate information for the diagnosis and treatment of patients.

### SUMMARY

The present disclosure introduces systems and methods for estimating perfusion parameters using medical imaging. In contrast to prior deconvolution-based techniques, perfusion parameters are estimated herein by recognizing data patterns using deep learning. In particular, as will be described, perfusion imaging data is utilized in a novel bi-input convolutional neural network (“bi-CNN”) framework to estimate perfusion parameter values.

In accordance with one aspect of the disclosure, a method for estimating perfusion parameters using medical imaging is provided. The method includes receiving a perfusion imaging dataset acquired from a subject using an imaging system, and assembling for a selected voxel in the perfusion imaging dataset a perfusion patch that extends in at least two spatial dimensions around the selected voxel and time. The method also includes correlating the perfusion patch with an arterial input function (AIF) patch corresponding to the selected voxel, and estimating at least one perfusion parameter for the selected voxel by propagating the perfusion patch and AIF patch through a trained convolutional neural network (CNN) that is configured to receive a pair of inputs. The method further includes generating a report indicative of the at least one perfusion parameter estimated.

In accordance with another aspect of the disclosure, a system for estimating perfusion parameters using medical imaging is provided. The system includes an input for receiving imaging data, and a processor programmed to carry out instructions for processing the imaging data received by the input. The instructions include accessing a perfusion imaging dataset acquired from a subject using an imaging system, selecting a voxel in the perfusion imaging dataset, and assembling for the selected voxel a perfusion patch extending in at least two spatial dimensions around the selected voxel and time. The instructions also include pairing the perfusion patch with an arterial input function (AIF) patch corresponding to the selected voxel, and estimating at least one perfusion parameter for the selected voxel by propagating the perfusion patch and AIF patch through a trained convolutional neural network (CNN) that is configured to receive a pair of inputs. The instructions further include generating a report indicative of the at least one perfusion parameter estimated. The system further includes an output for providing the report.

In accordance with yet another aspect of the present disclosure, a method for estimating perfusion parameters using medical imaging is provided. The method includes building a deep convolutional neural network (CNN) that is configured to receive a pair of inputs. The method also includes training the deep CNN using training data to generate a plurality of feature filters, and for each selected voxel in a perfusion imaging dataset, generating a perfusion patch and an arterial input function (AIF) patch. The method further includes applying the plurality of feature filters to the perfusion patch and AIF patch to estimate at least one perfusion parameter for each selected voxel.

The foregoing and other advantages of the invention will appear from the following description.

### BRIEF DESCRIPTION OF THE DRAWINGS

FIG. 1 is a graphical illustration showing deconvolution methods for obtaining perfusion parameters.

FIG. 2 is schematic diagram of an example system, in accordance with aspects of the present disclosure.

FIG. 3 is a flowchart setting forth steps of a process, in accordance with aspects of the present disclosure.

FIG. 4A is an illustration of a process, in accordance with aspects of the present disclosure.

FIG. 4B is an illustration of an example convolutional neural network, in accordance with aspects of the present disclosure.

FIG. 4C is an illustration of another example convolutional neural network, in accordance with aspects of the present disclosure.

FIG. 5 is a graphical illustration showing example learned temporal filters capturing signal changes along a time dimension for parameter estimation, in accordance with aspects of the present disclosure.

FIG. 6 is a graphical illustration comparing example perfusion maps estimated in accordance with aspects of the present disclosure relative to a ground truth.

### DETAILED DESCRIPTION

Several methods have been developed to estimate perfusion parameters using deconvolution. However, studies have found that deconvolution processes can introduce distortions that can influence the measurement of perfusion parameters and the decoupling of delay. In addition, perfusion parameter values can vary depending upon the different deconvolution methods being used, thereby leading to inconsistent outcome prediction. By contrast, the present disclosure introduces a novel approach that departs from such prior work. In particular, the present a system and method for estimating perfusion parameters based on identifying patterns (features) from inputted perfusion imaging data. In doing so, the present disclosure introduces a novel deep convolutional neural network (CNN) architecture that is configured to receive a pair of inputs, as will be described.

Conventionally, CNNs have been used to achieve state-of-the-art performance in difficult classification tasks, and involve learning feature filters from imaging. For instance, existing deep CNNs have been used to analyze images with multiple channels of information. In particular, deep CNNs are used to learn 3D detectors in order to extract features across 2D images with multiple color channels (e.g., red/green/blue channels). Such data-driven features have been shown to be effective in detecting local characteristics to improve classification.

The inventors have recognized that the power of CNNs may be adopted for perfusion parameter estimation. By utilizing a novel bi-input CNN architecture, it is demonstrated herein that important patterns may be extracted from perfusion imaging data in order to make accurate perfusion parameter estimations. To the best knowledge of the inventors, this is the first time that deep learning has been utilized to estimate perfusion parameters from medical imaging. As appreciated from descriptions herein, the present approach has the potential to improve the current quantitative analysis of perfusion images (e.g., increased robustness to noise), and may ultimately impact medical decision processes and improve outcomes for a variety of patients, such as patients at risk or suffering from stroke.

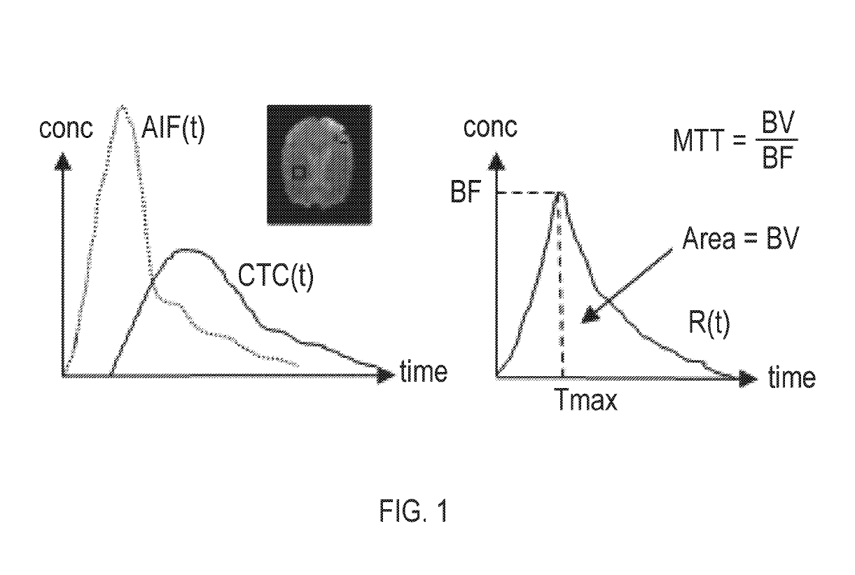

Turning now to FIG. 1, a block diagram of an example system 100, in accordance with aspects of the present disclosure, is shown. In general, the system 100 may include an input 102, a processor 104, a memory 106, and an output 108, and may be configured to carry out steps analyzing perfusion-weighted imaging in accordance with aspects of the present disclosure.

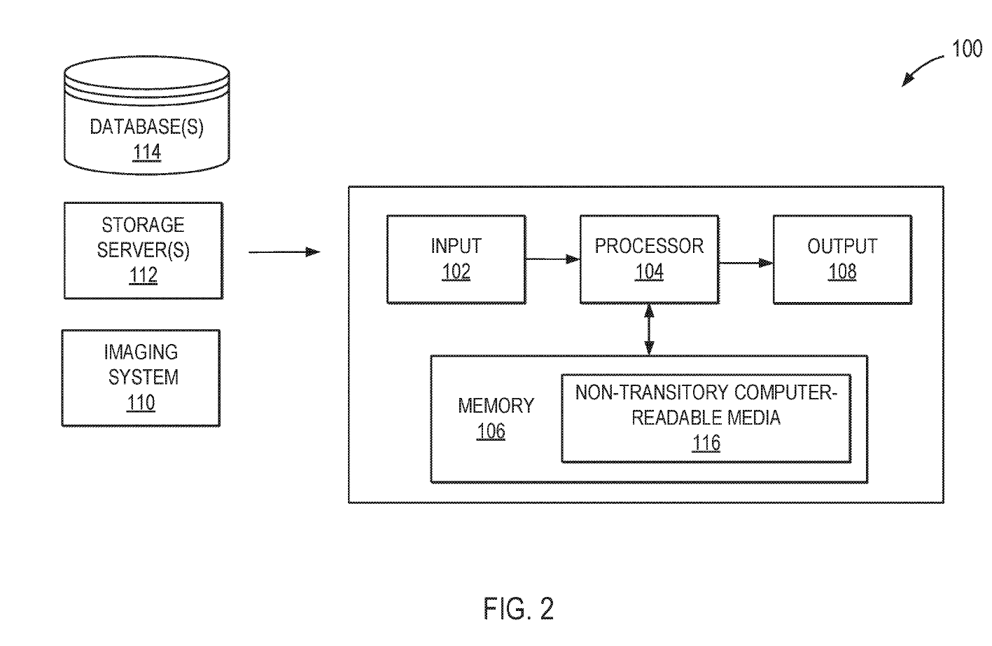

As shown in FIG. 2, the system 100 may communicate with one or more imaging system 110, storage servers 112, or databases 114, by way of a wired or wireless connection. In general, the system 100 may be any device, apparatus or system configured for carrying out instructions for, and may operate as part of, or in collaboration with various computers, systems, devices, machines, mainframes, networks or servers. In some aspects, the system 100 may be a portable or mobile device, such as a cellular or smartphone, laptop, tablet, and the like. In this regard, the system 100 may be any system that is designed to integrate a variety of software and hardware capabilities and functionalities, and capable of operating autonomously. In addition, although shown as separate from the imaging system 110, in some aspects, the system 100, or portions thereof, may be part of, or incorporated into, the imaging system 100, such as the magnetic resonance imaging (MRI) system described with reference to FIG. 8, or another imaging system.

Specifically, the input 102 may include different input elements, such as a mouse, keyboard, touchpad, touch screen, buttons, and the like, for receiving various selections and operational instructions from a user. The input 102 may also include various drives and receptacles, such as flash-drives, USB drives, CD/DVD drives, and other computer-readable medium receptacles, and be configured receive various data and information. To this end, the input 102 may also include various communication ports and modules, such as Ethernet, Bluetooth, or WiFi, for exchanging data and information with these, and other external computers, systems, devices, machines, mainframes, servers or networks.

In addition to being configured to carry out various steps for operating the system 100, the processor 104 may also be programmed to analyze perfusion imaging data, according to methods described herein. Specifically, the processor 104 may be configured to execute instructions, stored in a non-transitory computer readable-media 116, for example. Although the non-transitory computer readable-media 116 is shown in FIG. 2 as included in the memory 106, it may be appreciated that instructions executable by the processor 104 may be additionally or alternatively stored in another data storage location having non-transitory computer readable-media accessible by the processor 104.

The processor 104 may be configured to receive and process perfusion, and other imaging data, to generate a variety of information, including perfusion parameter estimates, or perfusion parameter maps. In particular, the perfusion imaging data may include perfusion-weighted imaging data acquired, for example, using an MRI system as described with reference to FIG. 8. Example perfusion-weighted imaging data include dynamic susceptibility contrast (DSC) imaging data, dynamic contrast enhanced (DCE) imaging data, arterial spin labeling imaging data, as well as other data. The processor 104 may also be programmed to direct acquisition of the perfusion imaging data. The perfusion imaging data may also include computed tomography (CT) data, positron emission tomography (PET) imaging data, ultrasound (US) imaging data, and others. The perfusion imaging data may include one dimensional (1D), two-dimensional (2D), three-dimensional (3D), and four-dimensional (4D) data, in the form of raw or processed data or images. In some aspects, the processor 104 may be programmed to access a variety of information and data, including perfusion imaging data, stored in the imaging system 110, storage server(s) 112, database(s) 114, PACS, or other storage location.

The processor 104 may also be programmed to preprocess the received or acquired imaging data, including perfusion imaging data, and other information. For example, the processor 104 may reconstruct one or more images using imaging data. In addition, the processor 104 may segment certain portions of an image or image set, for instance, by performing a skull-stripping or ventricle removal. The processor 104 may also select or segment specific target tissues, such as particular areas of a subject's brain, using various segmentation algorithms.

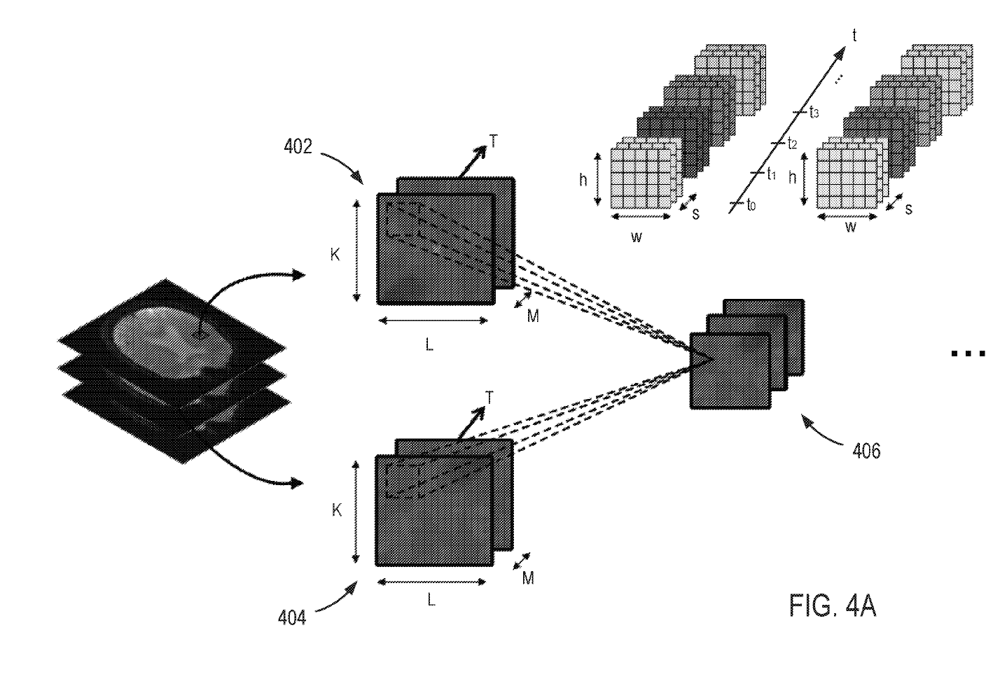

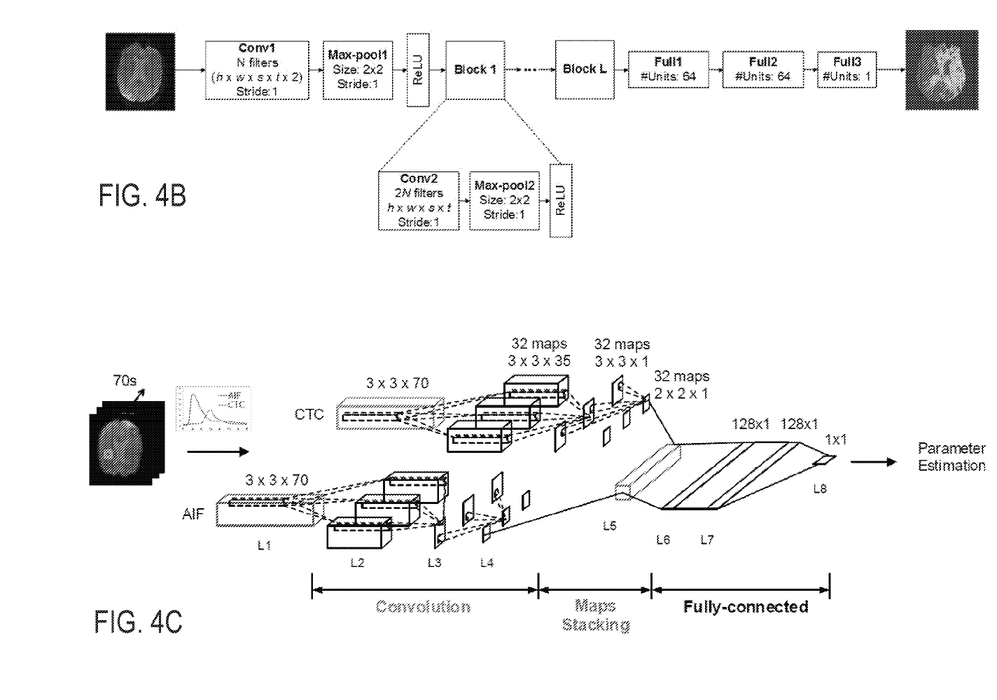

In accordance with the present disclosure, the processor 104 may be programmed to process perfusion imaging data to estimate one or more perfusion parameters. To do so, the processor 104 may select a number of voxels, or regions of interest, in a perfusion image or a perfusion image set and then generate various input patches using the selected voxels. Generated input patches may be two-dimensional (2D) extending in two spatial dimensions, three-dimensional (3D) extending in two spatial dimensions and one temporal dimension, or four-dimensional (4D) extending in three spatial dimensions and one temporal dimension. As shown in the example of FIG. 4A, a 4D input patch may be defined by a slice number s, a width w, a height h, and time t.

In some aspects, the processor 104 may use a provided perfusion imaging dataset to assemble perfusion patches and arterial input function patch (AIF). The perfusion imaging dataset may be a 3D imaging dataset or 4D imaging dataset, with the 3D imaging dataset including single images acquired at multiple time points and the 4D imaging dataset including multiple images or volumes acquired at multiple time points. In assembling a perfusion patch, neighboring voxels around a selected voxel may be used to construct the patch. As shown in the example of FIG. 4A, the perfusion patch may be a 4D input patch with spatial dimensions K, L, M, which need not be equal, and temporal dimension T. In assembling the AIF patch, the processor 104 may process the perfusion imaging dataset, using a singular value decomposition (SVD) technique for instance, to generate an AIF dataset. The processor 104 may then use the AIF dataset to generate an AIF patch corresponding to the perfusion patch. In some aspects, input patches may be cuboidal.

The generated patches may then be paired and propagated by the processor 104 through a trained bi-input CNN to estimate one or more perfusion parameter. Example bi-input CNN architectures are shown in FIGS. 4B and 4C. In particular, the network of FIG. 4B includes a first convolutional layer for paring detectors, followed by L blocks of convolution-pooling-ReLU layers, and then two fully connected layers before the output (estimated value). The value of L depends on the choices of h, w, s, and t. (Abbreviation: Conv=convolution, max-pool=max pooling, ReLU=rectified Linear Unit, Full=full-connected layer). Alternatively, the network of FIG. 4C includes a convolutional component, a stacking component, and a fully connected component. The processor 104 may select a number of voxels and repeat the above steps to estimate a plurality of perfusion parameters. In processing multiple voxels, the processor 104 may generate one or more images or perfusion parameter maps. Example perfusion parameters or parameter maps include blood volume (BV), blood flow (BF), mean transit time (MTT), time-to-peak (TTP), time-to-maximum (Tmax), maximum signal reduction (MSR), first moment (FM), and others. In some aspects, the processor 104 may also be configured to train a bi-input CNN using various images and information provided.

In some aspects, the processor 104 may be configured to identify various imaged tissues based on estimated perfusion parameters. For example, the processor 104 may identify infarct core and penumbra regions, as well as regions associated with abnormal perfusion. The processor 104 may be further programmed to determine a condition of the subject. For example, based on identified tissues or tissue regions, the processor 104 may determine a risk to the subject, such as a risk of infarction.

The processor 104 may also be configured to generate a report, in any form, and provide it via output 108. In some aspects, the report may include various raw or processed maps or images, or color-coded maps or images. For example, the report may include anatomical images, perfusion parameter maps including CBF, CBV, MTT, TPP, Tmax, Ktrans and other perfusion parameter maps. In some aspects, the report may indicate specific regions or tissues of interest, as well as other information. The report may further indicate a condition of the subject or a risk of the subject to developing an acute or chronic condition, such as a risk of infarction.

The biological derivation and definition of the four perfusion parameters of interest, namely cerebral blood volume (CBV), cerebral blood flow (CBF), MTT, Tmax, and their applications in stroke will now be described. However, it may be readily appreciated that the present disclosure is not limited to these perfusion parameters, nor applications related to stroke. Furthermore, the use of standard singular value decomposition (SVD) to obtain the residue function will also be described.

In MR perfusion imaging, a bolus of contrast dye is injected intravenously into a patient during continuous imaging, allowing for the concentration of contrast to be measured for each voxel over time as the bolus is disseminated throughout the body. Using this temporal data, model-based perfusion parameters may be calculated and used to create parameter maps of the brain following a stroke, for example. Such parameter maps are useful for identifying tissue that can be potentially salvageable with treatment.

Typically, tissue perfusion is modeled by the Indicator-Dilution theory, where the measured tissue concentration time curve (CTC) of a voxel is directly proportional to the convolution of the arterial input function (AIF) and the residue function (R), as scaled by cerebral blood flow (CBF). This model follows the principle of the conservation of mass, meaning that the amount of contrast entering the voxel is equal to the sum of the contrast leaving the voxel and the contrast within the voxel. To obtain the perfusion parameters, the residue function (R) may be derived by applying a Singular Value Decomposition (SVD) technique and the following expression:

CTC(t)=CBF∫₀^{τ}AIF(t)R(t−τ)dτ, (1)

In perfusion images, CTC(t) and AIF(t) can be observed from the raw signals. To obtain R(t) by SVD, Eqn. 1 may be first discretized to:

ctc(tⱼ)=Δt·CBF·Σᵢ₌₀^{j}AIF(tᵢ)·R(tⱼ−tᵢ), (2)

where Δt is the sampling frequency. Eqn. 2 may then be formulated as an inverse matrix problem:

$$\begin{matrix}[ \begin{matrix}ctc ( {t}_{0} ) \\ ctc ( {t}_{3} ) \\ ⋮ \\ ctc ( {t}_{N - 1} )\end{matrix} ] = Δ t [ \begin{matrix}AIF ( {t}_{0} ) & 0 & … & 0 \\ AIF ( {t}_{1} ) & AIF ( {t}_{0} ) & … & 0 \\ ⋮ & ⋮ & ⋱ & ⋮ \\ AIF ( {t}_{N - 1} ) & AIF ( {t}_{N - 2} ) & … & AIF ( {t}_{0} )\end{matrix} ] \times [ \begin{matrix}R ( {t}_{0} ) \\ R ( {t}_{1} ) \\ ⋮ \\ R ( {t}_{N - 1} )\end{matrix} ] \cdot CBF , \\ or & ( 3 ) \\ c = A \cdot b , & ( 4 )\end{matrix}$$

where c represents the CTC(t), A represents the AIF(t), and b represents the R(t) (constants are not shown for simplification). Using SVD, A can be decomposed as follows:

A=U·S·V^{T}, (5)

A⁻=V·W·U^{T}, (6)

where U and V are orthogonal matrices and S is a non-negative square diagonal matrix, W=1/S along the diagonals and zero elsewhere. Then, b, or R(t), can be obtained as following

b=V·W·U^{T}·c. (7)

Four parameters, namely CBV, CBF, MTT, Tmax can be obtained from R(t). CBV describes the total volume of flowing blood in a given volume of a voxel. It is equal to the area under the curve of R(t). CBF describes the rate of blood delivery to the brain tissue within a volume of a voxel, and is the constant scaling factor of the ratio between the CTC and the convolution of the arterial input function (AIF) and the residue function in Eqn. 1. It is equal to the maximum value of the residue function. By the Central Volume Theorem, CBV and CBF can be used to derive MTT, which represents the average time it takes the contrast to travel through the tissue volume of a voxel. Tmax is the time point where the R(t) reaches its maximum. It approximates the time needed for the bolus to arrive at the voxel. The mathematical expressions of these parameters are listed in the following:

$$CBV = {\int }_{0}^{∞} R ( t ) dt , \\ CBF = max ( R ( t ) ) , \\ MTT = \frac{CBV}{CBF} , \\ T max = arg {max}_{t} ( R ( t ) ) . \qquad ( 8 )$$

These parameters are important for characterizing underlying brain tissue, for instance. Specifically, a patient with arterial occlusion and ischemic stroke normally has a substantial drop in CBF and CBV, and a higher Tmax in the affected brain volume distal to the blood vessel blockage. Initially, affected brain volumes may be salvageable, but irreversible damage can occur over several hours due to insufficient blood supply. Thresholds have been established for these perfusion parameters that define the volume of dead tissue core and the under-perfused but potentially salvageable tissue.

In estimating these perfusion parameter for a selected voxel, given CTC and AIF measurements, a pattern recognition model in the form of a novel bi-input convolutional neural network (bi-CNN), which takes the two inputs (CTC, AIF) may be used. In some aspects, separate bi-CNNs m trained to estimate each perfusion parameter. The overall estimation task may be defined as:

ν=f(AIF,CTC), (9)

where v is the estimated value, and f(⋅) is the bi-CNN with the trained weights. The bi-CNN may be trained with thousands of training patches to learn important features from the input data in order to make an accurate approximation.

With regard to the example of FIG. 4C, a bi-CNN architecture, in accordance with aspects of the present disclosure, may include three components: (1) convolution, (2) maps stacking, and (3) fully-connected. In the convolution, a CTC and its AIF may be convolved independently via multiple convolutional layers (i.e., two convolution chains), where temporal filters are learned. Each convolution chain may follow a denoising architecture that attempts to remove artifacts (e.g., noise, distortion) that are often seen in the input perfusion signals. This is advantageous for identifying fine-grained features from CTC and AIF signals that help estimation. As suggested previously, a simple signal with artifacts can be model as follows:

y=x*k. (10)

where y is an observed 1D signal (instead of a 2D image), x is the original artifact-free signal, and k is a convolution kernel accounting for artifacts. A Fourier transform operator, F(⋅), with Tikhonov regularizer, may then be applied, with x expressed as:

$$χ = {F}^{- 1} ( \frac{1}{F ( k )} \{ \frac{{\vert F ( k ) \vert}^{2}}{{\vert F ( k ) \vert}^{2} + \frac{1}{SNR}} \} ) * y = {k}^{*} * y , \qquad ( 11 )$$

where SNR is the signal to noise ratio and k* is the pseudo inverse kernel. The new representation of x can be further expanded into a matrix representation by the kernel separability theorem, where k* is decomposed into k*=U·S·VT. This leads to a new representation of x:

x=k**y=Σⱼsⱼ·uⱼ*(vⱼ^{T}*y), (12)

where uⱼ and vⱼ are the j^{th} columns of U and V respectively, and sⱼ is the j^{th} singular value. This new expression shows that the original artifact-free signal, x, can be obtained via the weighted sum of separable 1D filters. This can lead to the design of a convolution chain where two separated 1D convolutions are performed (L1 to L2, and L2 to L3), with filter size of 1×1×36 and 1×1×35 respectively, for example. A convolutional layer (L3 to L4) can then be added after the denoising architecture to learn filters for detecting the spatial contributions of neighboring voxels. The output feature maps of the convolution chains may then be stacked together in the maps stacking layer (L5), resulting in a matrix with a size of 64×2×2×1, for example. It is then connected to two fully-connected layers where hierarchical features are learned to correlate the AIF and CTC derived features. The output of the network (L8) is the estimated parameter value. The training optimization of the network may then be configured to obtain network weights, Θ, that minimize the mean squared loss between the true value, V, and the estimated value, \hat{V}(Θ), across the samples with size n:

$$arg {min}_{Θ} loss = \frac{1}{n} {\sum }_{i = 1}^{n} {( {V}_{i} - {\hat{V}}_{i} ( Θ ) )}^{2} , \qquad ( 13 )$$

A bi-CNN architecture, in accordance with aspects of the present disclosure, may include a max-pooling layer (with a max operator), which helps identifying maximum values. The max-pooling layer may be inserted into L3 to replace the second convolutional layer in each convolutional chain for bi-CNNs of CBF. The size of the maxpooling layer may be set to 1×1×35, for example, to maintain the size consistency across the rest of the network.

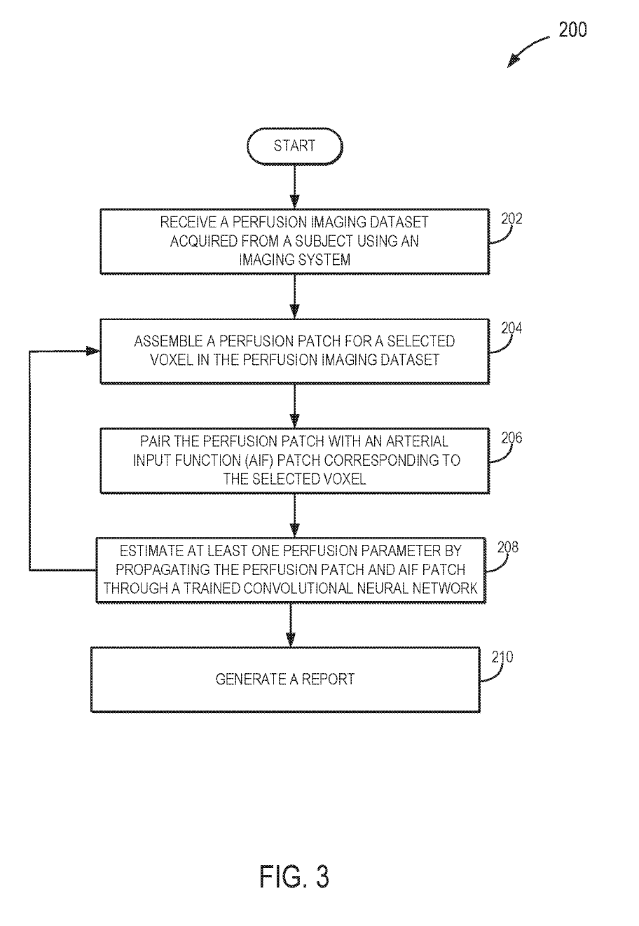

Turning now to FIG. 3, a flowchart setting forth steps of a process 200, in accordance with aspects of the present disclosure is shown. The process 200 may be carried out using any suitable system, device or apparatus, such as the system 100 described with reference to FIG. 1. In some aspects, the process 200 may be embodied in a program or software in the form of instructions, executable by a computer or processor, and stored in non-transitory computer-readable media.

The process 200 may begin at process block 202 with receiving a perfusion imaging dataset acquired from a subject. The perfusion imaging dataset may be a three-dimensional (3D) or four-dimensional (4D) perfusion imaging dataset. In particular, the 4D perfusion imaging data set may include a time-resolved series of images, with one or more images in the series being associated with a different time points or time periods. Alternatively, the perfusion imaging dataset may include raw imaging data acquired at one or more time points or time periods. To this end, a reconstruction may be carried out at process block 202, as well as other processing steps, as described. In some aspects, anatomical images or data may also be received at process block 202 in addition to the perfusion imaging dataset.

In some implementations, the perfusion imaging dataset received at process block 202 may include perfusion-weighted imaging data acquired using an MRI system. For instance, perfusion-weighted imaging data may be acquired using a perfusion acquisition, such as dynamic susceptibility contrast (DSC) or dynamic contrast enhanced (DCE) pulse sequence carried out during the administration of an intravascular contrast agent to the subject. In addition, perfusion-weighted imaging data may also be acquired without the use of contrast agents, for instance, using an arterial spin labeling (“ASL”) pulse sequence. In addition, the perfusion imaging dataset received at process block 202 may also include other perfusion imaging data, such as imaging data acquired using a CT system using different contrast agents and techniques. As described data, images and other information may be accessed from a memory, database, or other storage location. Alternatively, or additionally, a data acquisition process may be carried out at process block 202 using an imaging system, such as an MRI system.

Then, at process block 204, a perfusion patch may be assembled for a selected voxel using the received perfusion imaging dataset. The perfusion patch may be paired with an AIF patch corresponding to the selected voxel at process block 206, where the AIF patch is generated using the perfusion imaging dataset. The patches may then be propagated through a trained CNN to estimate at least one perfusion parameter for the selected voxel, as indicated by process blocks 208 Example perfusion parameters include blood volume (“BV”), blood flow (“BF”), mean transit time (MTT), time-to-peak (TTP), time-to-maximum (Tmax), maximum signal reduction (MSR), first moment (FM), Ktrans and others. As indicated in FIG. 3, process blocks 204 through 208 may be repeated a number of times, each time selecting a different voxel. In this manner, a plurality of perfusion parameters can be estimated. These can then be used to generate one or more perfusion parameter maps.

Training a CNN is illustrated in the example of FIG. 4A. Specifically, a perfusion patch 402 is coupled with its corresponding AIF patch 404, with a size of K×L×N×t, where M is the number of brain slices, K is the height, L is the width, and T is the time (i.e. the number of sequences in a perfusion-weighted image). Pairs of 4D detectors (h×w×s×t) are learned to convolve each perfusion patch and AIF patch together, generating N feature maps 406 in the first convolution layer. The feature maps are the inputs to the next layer. The CNN may be constructed to accept spatio-temporal perfusion data with corresponding AIF data in order to learn paired convolved features. These features represent the spatio-temporal correlations between the perfusion patch and the AIF patch. Such correlations may then be further analyzed in subsequent layers to learn hierarchical features predictive of perfusion parameters.

The present approach extends the typical convolutional layer so that multiple pairs of 4D feature detectors can be learned at the first layer and multiple 4D feature detectors can be learned in the L layers instead of common 3D feature detectors (FIG. 4B) Through learning these 4D feature detectors, correlations between the arterial input function patch and the perfusion patch are extracted, as well as elementary features such as curvature, endpoints, and corners along time from the input images. Convolutional layers learn multiple 4D feature detectors that capture hierarchical features from the previous input layer and generate useful feature maps that are used as inputs for the next layer. In pooling layers, local groups of input values are combined. Non-linear layers are inserted between each convolutional and pooling layers to introduce non-linearity to the network. A fully-connected layer contains output neurons that are fully connected to input neurons. The last fully-connected layer contains rich representations that characterize a voxel input signal and these features can be used in a non-linear unit to estimate a perfusion parameter. Weights in the network may be learned using a variety of optimization techniques, including stochastic gradient descent via backpropagation.

Referring again to FIG. 3, a report may then be generated at process block 210. The report may be in any form, and provide various information. In some aspects, the report may include various raw or processed maps or images, or color-coded maps or images. For example, the report may include anatomical images, maps of CBF, CBV, MTT, TPP, Tmax, Ktrans and other perfusion parameters. In some aspects, the report may indicate or highlight specific regions or tissues of interest, as well as provide other information. The report may further indicate a condition of the subject or a risk of the subject to developing an acute or chronic condition, such as a risk of infarction. To this end, generated perfusion parameters, maps or images may be analyzed to determine the condition or tissue types, or tissue regions.

The above-described system and method may be further understood by way of example. The following example is offered for illustrative purposes only, and is not intended to limit the scope of the present invention in any way. Indeed, various modifications of the invention in addition to those shown and described herein will become apparent to those skilled in the art from the foregoing description and the following examples and fall within the scope of the appended claims. For example, certain arrangements and configurations are presented, although it may be understood that other configurations may be possible, and still considered to be well within the scope of the present invention.

### Example

Perfusion magnetic resonance (MR) images are often used in the assessment of acute ischemic stroke to distinguish between salvageable tissue and infarcted core. Deconvolution methods such as singular value decomposition have been used to approximate model-based perfusion parameters from these images. However, studies have shown that these existing deconvolution algorithms can introduce distortions that may negatively influence the utility of these parameter maps. In the past, limited work was done on utilizing machine learning algorithms to estimate perfusion parameters. In this work, a novel bi-input convolutional neural network (bi-CNN) is introduced to approximate four perfusion parameters without using an explicit deconvolution method. These bi-CNNs produced good approximations for all four parameters, with relative average root-mean-square errors (ARMSEs)≤5% of the maximum values. The utility of the estimated perfusion maps is further demonstrated by quantifying the salvageable tissue volume in stroke, with more than 80% agreement with the ground truth. These results show that deep learning techniques are a promising tool for perfusion parameter estimation without need for applying a standard deconvolution process.

Dataset:

MR perfusion data was collected retrospectively for a set of 11 patients treated for acute ischemic stroke at UCLA. The ground truth perfusion maps (CBV, CBF, MTT, Tmax) and AIFs were generated using bSVD in the sparse perfusion deconvolution toolbox and the ASIST-Japan perfusion mismatch analyzer respectively. All the perfusion images were interpolated to have a consistent 70 s time interval for bi-CNNs. The ranges of CBV, CBF, MTT, and Tmax values were between 0-201 ml/100 g, 0-1600 ml/100 g/min, 0-25.0 s, and 0-69 s (Tmax was clipped at 11 s because there were too few examples beyond this value) respectively. Since unequal sampling of the training data can lead to biased prediction, each perfusion parameter value was grouped into ten bins, and equal sized training samples were drawn from each bin. This resulted in four sets of training data (CBV, CBF, MTT, Tmax), with sizes of 91,950, 97,110, 87,080, and 74,850 respectively.

CNN Configuration and Implementation:

The overview of the bi-CNN is shown in FIG. 4B. A training example consisted of a pair of input patches: CTC and its AIF, with a size of 3×3×70. Each convolution chain consisted of three convolutional layers where 32 maps were learned (with zero-padding and a stride of 1). A non-linear rectified linear unit (ReLU) layer was attached to every convolutional layer and fully-connected layer (except for the max-pooling layer). It may be noted that the present architecture included two features distinct from traditional CNN configurations providing optimized performance of the model. First, dropout was not included in the fully-connected layers because decreased performance was observed during validation. This may be due to the nature of the problem of parameter estimation (i.e., estimating a continuous value versus predicting a categorical label), where every output unit may contribute (to some degree) to the estimated value. Second, the initial learning rates were different for different parameter estimations. The training losses were observed to easily explode when the learning rate was too high, especially for perfusion parameters with high maximum values (e.g., max(CBF)=1600). Therefore, the initial learning rates for CBV, CBF, MTT, and Tmax were 0.0005, 0.00005, 0.005, 0.005 respectively, with a learning rate decay of 1e-8, 1e-9, 1e-7, 1e-7 respectively.

The bi-CNN was trained with batch gradient descent (batch size: 50; epochs: 10) and backpropagation. A momentum of 0.9 were used. A heuristic was applied to improve the learning of deep CNN weights, where the learning rate was divided by 10 when the validation error rate stopped improving with the current learning rate. This heuristic was repeated three times. The deep CNN was implemented in Torch7, and the training was done on a NVIDIA Tesla K40 GPU.

Evaluation:

The performance of the bi-CNN estimators was evaluated by leave-one-patient-out cross-validation (i.e., training was performed excluding data from one patient and then evaluating the results on that held-out patient). The average root-mean-square error (ARMSE) of validations was calculated using following definition

$$ARMSE = \frac{1}{{n}_{T}} {\sum }_{j = 1}^{{n}_{T}} \sqrt{\frac{1}{{s}_{j}} {\sum }_{i = 1}^{{s}_{j}} {( {V}_{i , j} - {\hat{V}}_{i , j} )}^{2}} , \qquad ( 14 )$$

where n_{T} is the total number of patients, V is the ground truth value, \hat{V} is the estimated value, and sⱼ is the number of samples.

The utility of the bi-CNN was also demonstrated by comparing the salvageable tissue binary masks generated from the bi-CNN and the ground truth perfusion maps. Published CBF and Tmax thresholds were used to define the salvageable tissue binary masks. The similarity between these masks (the ground truth mask, A, and the estimated mask, B) was calculated using the Dice coefficient

$$Dice ( A , B ) = 2 \frac{\vert A ⋂ B \vert}{\vert A \vert + \vert B \vert} , \qquad ( 15 )$$

A value of 0 indicates no overlap, and a value of 1 indicates perfect similarity (i.e., B=A). A good overlap between masks is generally considered to have occurred when the Dice coefficient is larger than 0.7.

Results and Discussion:

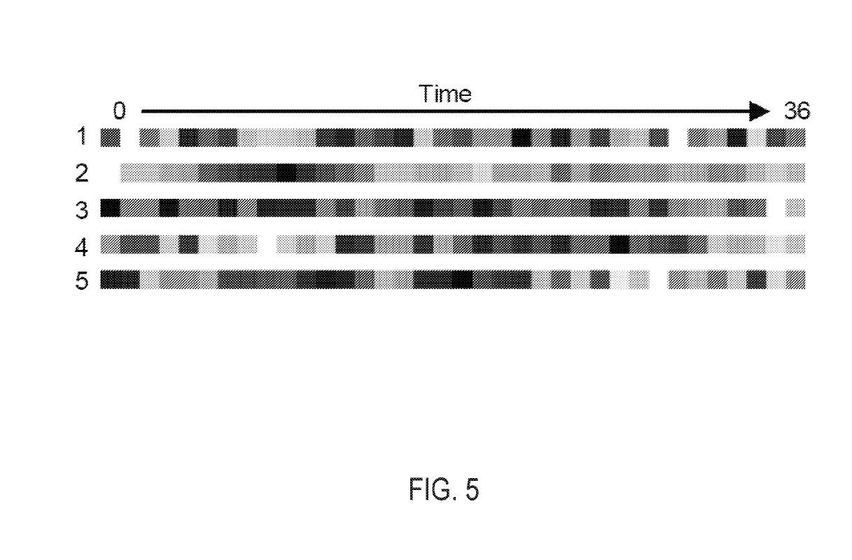

FIG. 5 shows some examples of learned convolutional filters from the first layer of the CTC convolution chain. Each row represents a 1×1×36 temporal filter and each column is a unit filter at a time point. As can be seen, these filters capture high signals (white) and low signals (black) at different time points, which helps the fine-grained temporal feature detections from the source signals. This is important to identify features for accurate parameter estimation. Using such learned temporal filters, the bi-CNNs achieved an ARMSE of 4.80 ml/100 g, 27.4 ml/100 g/min, 1.18 s, 1.33 s for CBV, CBF, MTT, and Tmax respectively, which are equivalent to 2.39%, 1.71%, 4.72%, and 1.19% of the individual perfusion parameter's maximum value. The small ARMSE results showed that the bi-CNNs are capable of learning feature filters to approximate perfusion parameters from CTCs and AIFs without using standard deconvolution.

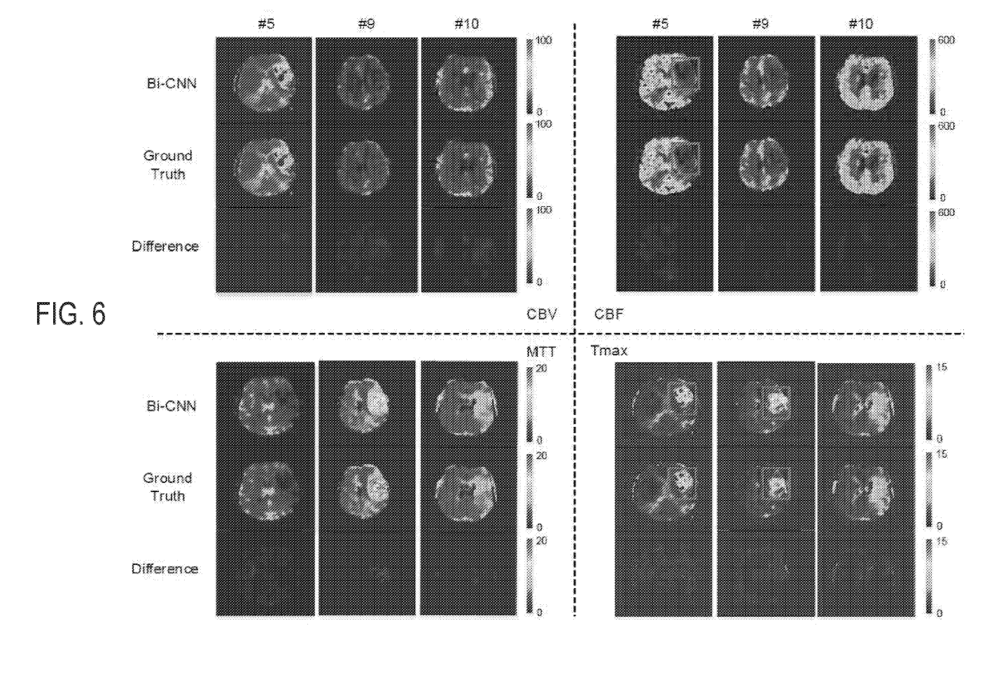

Examples of estimated perfusion maps are shown in FIG. 6. All of the estimated perfusion maps (CBV, CBF, MTT, and Tmax) showed good alignment with the ground truth and hypoperfusion (i.e. less blood flow or delayed Tmax) could be observed visually from some of the estimated maps (red boxes). The differences between the estimated maps and the ground truth were minimal. To further verify the usability of the estimated perfusion maps, a CBF cutoff of 50.2 ml/100 g/min and a Tmax cutoff of 4 s were used to generate the salvageable tissue masks from the ground truth and the estimated perfusion maps (FIG. 6). The average Dice coefficients for the CBF and Tmax masks were 0.830±0.109 and 0.811±0.071 respectively, showing good overlap between the ground truth masks and the estimated masks. These results show that the bi-CNN, in accordance with the present disclosure, can generate useful masks for salvageable tissue approximation.

The performance of the present bi-CNN, which is a machine learning approach different from standard deconvolution, is based on the amount of available training data. With more cases, larger networks with more epochs can be trained to learn the variability embodied by additional patients, which could potentially improve the performance. Second, the present bi-CNN may be evaluated using digital phantoms, which is a more accurate source of ground truth. Third, it is envisioned that an optimal patch size can be obtained for the parameter estimation, with more spatial context information may boost the performance of the voxel-wise estimation, for instance. Finally, using the current implementation of bi-CNNs to generate an estimated perfusion map required more computational time than standard deconvolution (˜5× slower). To address this, batch and multi-GPU processing may be implemented in order to shorten the map generation time so that it is practical to apply the models clinically.

In summary, a novel approach for perfusion parameter estimation using a bi-input convolutional neural network is introduced herein. Results showed that the patch-based bi-CNN model is capable of estimating four perfusion parameters in stroke patients without using a standard deconvolution methods. The estimated perfusion maps can be used to generate binary masks that are representative of the salvageable tissue. This model can potentially be extended to other disease domains in which perfusion imaging is used, such as cancer.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, computer processor, central processing unit (CPU), and computer are used synonymously to denote a device capable of executing the instructions and communicating with input/output interfaces and/or peripheral devices, and that the terms processor, computer processor, CPU, and computer are intended to encompass single or multiple devices, single core and multicore devices, and variations thereof. Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

In addition to any other claims, the applicant(s)/inventor(s) claim each and every embodiment of the technology described herein, as well as any aspect, component, or element of any embodiment described herein, and any combination of aspects, components or elements of any embodiment described herein.

Features suitable for such combinations and sub-combinations would be readily apparent to persons skilled in the art upon review of the present application as a whole. The subject matter described herein and in the recited claims intends to cover and embrace all suitable changes in technology.

What is claimed is:

1. A method for estimating perfusion parameters using medical imaging, the method comprising:
(a) receiving a perfusion imaging dataset acquired from a subject using an imaging system;
(b) assembling for a selected voxel in the perfusion imaging dataset a perfusion patch that extends in at least two spatial dimensions around the selected voxel and time;
(c) correlating the perfusion patch with an arterial input function (AIF) patch corresponding to the selected voxel;
(d) estimating at least one perfusion parameter for the selected voxel by propagating the perfusion patch and AIF patch through a trained convolutional neural network (CNN) that is configured to receive a pair of inputs; and
(e) generating a report indicative of the at least one perfusion parameter estimated.

2. The method of claim 1, wherein the perfusion imaging dataset comprises a three-dimensional (3D) or four-dimensional (4D) perfusion imaging dataset.

3. The method of claim 1, wherein the perfusion imaging dataset is acquired using a magnetic resonance imaging (MRI) system performing a dynamic susceptibility contrast (DSC) technique, a dynamic contrast enhanced (DCE) technique or an arterial spin labeling technique.

4. The method of claim 1, wherein the trained CNN comprises a convolutional component, a stacking component, and a fully connected component.

5. The method of claim 1, wherein the method further comprises generating the AIF patch by applying a singular value decomposition (SVD) technique using the perfusion imaging dataset.

6. The method of claim 1, wherein the at least one perfusion parameter is a blood volume (BV), a blood flow (BF), a mean transit time (MTT), a maximum time (Tmax), a time to peak (TTP), a maximum signal reduction (MSR), a first moment (FM), or a combination thereof.

7. The method of claim 1, wherein the method further comprises repeating steps (b) through (d) for a plurality of selected voxels to estimate a plurality of perfusion parameters.

8. The method of claim 7, wherein the method further comprises constructing a perfusion map using the plurality of perfusion parameters.

9. A system for estimating perfusion parameters using medical imaging, the system comprising:
an input for receiving imaging data;
a processor programmed to carry out instructions for processing the imaging data received by the input, the instructions comprising:
i) accessing a perfusion imaging dataset acquired from a subject using an imaging system;
ii) selecting a voxel in the perfusion imaging dataset;
iii) assembling for the selected voxel a perfusion patch extending in at least two spatial dimensions around the selected voxel and time;
iv) pairing the perfusion patch with an arterial input function (AIF) patch corresponding to the selected voxel;
v) estimating at least one perfusion parameter for the selected voxel by propagating the perfusion patch and AIF patch through a trained convolutional neural network (CNN) that is configured to receive a pair of inputs;
vi) generating a report indicative of the at least one perfusion parameter estimated; and
an output for providing the report.

10. The system of claim 9, wherein the perfusion imaging dataset comprises a three-dimensional (3D) or four-dimensional (4D) perfusion imaging dataset.

11. The system of claim 9, wherein the processor is further configured to propagate the perfusion patch and AIF patch through a trained CNN comprising a convolutional component, a stacking component, and a fully connected component.

12. The system of claim 9, wherein the processor is further configured to generate the AIF patch by applying a singular value decomposition (SVD) technique using the perfusion imaging dataset.

13. The system of claim 9, wherein the processor is further configured to estimate a blood volume (BV), a blood flow (BF), a mean transit time (MTT), a maximum time (Tmax), a time to peak (TTP), a maximum signal reduction (MSR), a first moment (FM), or a combination thereof.

14. The system of claim 9, wherein the processor is further configured to repeat steps (ii) through (v) to select a plurality of voxels and estimate a plurality of perfusion parameters.

15. The system of claim 9, wherein the processor is further configured to construct a perfusion map using the plurality of perfusion parameters.

16. A method for estimating perfusion parameters using medical imaging, the method comprising:
building a deep convolutional neural network (CNN) that is configured to receive a pair of inputs;
training the deep CNN using training data to generate a plurality of feature filters;
for each selected voxel in a perfusion imaging dataset, generating a perfusion patch and an arterial input function (AIF) patch; and
applying the plurality of feature filters to the perfusion patch and AIF patch to estimate at least one perfusion parameter for each selected voxel.

17. The method of claim 16, wherein the trained CNN comprises a convolutional component, a stacking component, and a fully connected component.

18. The method of claim 16, wherein the method further comprises training the deep CNN using a batch gradient descent and a backpropagation technique.

19. The method of claim 16, wherein the method further comprises estimating a blood volume (BV), a blood flow (BF), a mean transit time (MTT), a maximum time (Tmax), a time to peak (TTP), a maximum signal reduction (MSR), a first moment (FM), or a combination thereof.

20. The method of claim 16, wherein the method further comprises constructing a perfusion map using a plurality of perfusion parameters corresponding to multiple voxels.
